(19) United States
(12) Patent Application Publication
(10) Pub. No.: US 2009/0203716 A1
(43) Pub. Date: Aug. 13, 2009

(54) Pyrimidine low molecular weight ligands for modulating hormone receptors

(75) Inventors: Marvin C. Gershengorn, Washington, DC (US); Susanne Neumann, Bethesda, MD (US); Craig J. Thomas, Gaithersburg, MD (US); Holger Jaeschke, Suepitz (DE); Susanna Moore, Rockville, MD (US); Gerd Krause, Berlin (DE); Bruce Raaka, Rockville, MD (US); Ralf Paschke, Markleeberg (DE); Gunnar Kleinau, Berlin (DE)

Correspondence Address: KLARQUIST SPARKMAN, LLP, 121 S.W. SALMON STREET, SUITE #1600, PORTLAND, OR, 97204-2988, US

(73) Assignees: The Government of the United States of America as represented by the Secretary of the; Department of Health and Human Services

(21) Appl. No.: 12/291,932

(22) Filed: Nov. 14, 2008

Related U.S. Application Data
(63) Continuation-in-part of application No. PCT/US2007/011951, filed on May 17, 2007.
(60) Provisional application No. 60/801,370, filed on May 17, 2006.

(51) Int. Cl. A61K 31/519; C07D 495/04

(52) U.S. Cl. 514/260.1; 544/278

(57) ABSTRACT

Disclosed herein are small molecule modulators hormone receptors, including agonists and antagonists of luteinizing hormone/choriogonadotropin, follicle stimulating hormone and thyroid stimulating hormone receptors. Exemplary disclosed compounds include those of the formula

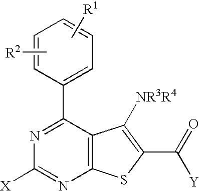

wherein X is —S(O)ₙR⁵;
n is 0, 1 or 2;
Y is —OR⁶ or —NR⁷R⁸
R¹ and R² independently are selected from optionally substituted lower aliphatic, alkoxy, aralkyl, halogen, H and —OR⁵, wherein R⁵ is selected from lower alkyl, H, aralkyl, acyl, alkoxycarbonyl and aminocarbonyl;
R³ and R⁴ independently are selected from acyl, alkoxycarbonyl, aminocarbonyl, aralkyl, H, lower alkyl and cycloalkyl;
R⁵ is selected from lower alkyl, aralkyl, cycloalkyl and haloalkyl;
R⁶ is selected from H, lower alkyl and aralkyl;
R⁷ and R⁸ independently are selected from H, lower alkyl, aralkyl and cycloalkyl.

### CROSS REFERENCE TO RELATED APPLICATION

This application is a continuation-in-part of International Application No. PCT/US2007/011951, filed May 17, 2007, which claims the benefit of the earlier filing date of U.S. Provisional Patent Application No. 60/801,370 filed May 17, 2006, both of which are incorporated herein by reference in their entireties.

### FIELD

This disclosure concerns hormone receptor modulating compounds and methods for their use.

### BACKGROUND

Luteinizing hormone/choriogonadotropin (LH/CG), follicle-stimulating hormone (FSH) and thyroid-stimulating hormone (TSH) are heterodimeric glycoprotein hormones that regulate reproduction and thyroid homeostasis. LH is responsible for ovulation induction in women and controls testosterone production in men. FSH causes ovarian follicle maturation in women and is involved in spermatogenesis in men. TSH is involved in the growth and function of thyroid follicular cells. Cellular responses to all three glycoprotein hormones are mediated via distinct seven transmembrane-spanning receptors, for example, the LHCG, FSH and TSH receptors. Each receptor is characterized by an elongated extracellular domain distinguished by several leucine-rich motifs that are involved in recognition and binding of the large glycoprotein hormones. The seven-transmembrane helices of each receptor are noteworthy because of their high degree of homology.

Disruption of physiological regulation of LHCG receptor, FSH receptor and TSH receptor by diverse pathogenic mutations has been implicated in a number of human diseases. The specific and potent control of these multifunctioning receptors could provide important therapeutic advancements. LH and FSH are currently used clinically for the treatment of infertility. Recombinant TSH is used in the diagnostic screen for thyroid cancer. TSH receptor agonists and antagonists may well have utility in the diagnosis and treatment of thyroid cancer, respectively. The development of small molecule modulators of LHCG receptor and FSH receptor has also been pursued with varying degrees of success.

### SUMMARY

Disclosed herein are modulators of hormone receptors, including agonists and antagonists of the luteinizing hormone receptor, follicle stimulating hormone receptor and thyroid-stimulating hormone receptor. Examples of such hormone receptor modulators include those of the formula

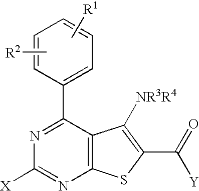

wherein X is —S(O)ₙR⁵;

n is 0, 1 or 2;

Y is —OR⁶ or —NR⁷R⁸

R¹ and R² independently are selected from optionally substituted lower aliphatic, alkoxy, aralkyl, halogen, H and —OR⁵, wherein R⁵ is selected from lower alkyl, H, aralkyl, acyl, alkoxycarbonyl and aminocarbonyl;

R³ and R⁴ independently are selected from acyl, alkoxycarbonyl, aminocarbonyl, aralkyl, H, lower alkyl and cycloalkyl;

R⁵ is selected from lower alkyl, aralkyl, cycloalkyl and haloalkyl;

R⁶ is selected from H, lower alkyl and aralkyl; and

R⁷ and R⁸ independently are selected from H, lower alkyl, aralkyl and cycloalkyl.

According to another embodiment, there are provided compounds that are antagonists of the thyroid-stimulating hormone receptor of the formula

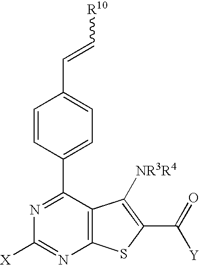

wherein R¹⁰ is —S(O)ₙR⁵ or —OR⁹, wherein R⁵ is selected from lower alkyl, H, aralkyl, acyl, alkoxycarbonyl and aminocarbonyl, n is 0, 1 or 2, and R⁹ is selected from acyl, alkoxycarbonyl, aminocarbonyl, aralkyl, H, lower alkyl and cycloalkyl;

X is —S(O)ₙR⁵; wherein R⁵ is selected from lower alkyl, H, aralkyl, acyl, alkoxycarbonyl and aminocarbonyl, and n is 0, 1 or 2;

Y is —OR⁶ or —NR⁷R⁸, wherein R⁶ is selected from H, lower alkyl and aralkyl, and R⁷ and R⁸ independently are selected from H, lower alkyl, aralkyl and cycloalkyl; and

R³ and R⁴ independently are selected from acyl, alkoxycarbonyl, aminocarbonyl, aralkyl, H, lower alkyl and cycloalkyl.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates the analysis of compounds 3 and 20 at both the TSH receptor and the LHCG receptor, comparing activation of TSH receptor and the LHCG receptor by compounds 3 and 20 relative to basal activities of both receptors.

FIG. 2 illustrates full concentration analyses of compounds 3, 5, and 7 at TSH receptor and LHCG receptor, with the data presented as mean±SEM of two independent experiments, each performed in duplicate.

FIG. 3 illustrates the antagonistic activity of compound 52 at TSHR and LHCGR. Intracellular cAMP accumulation was determined in response to increasing concentrations of compound 52. EC₅₀ concentrations of native ligands were as follows: TSH, 1.8 nM; LH, 0.34 nM.

FIG. 4 illustrates that compound 52 activates TSHR mutants Y7.42A and M9 in contrast to TSHR. Intracellular cAMP accumulation was determined without ligands (basal) or in response to 30 μM of compound 52.

FIG. 5 illustrates that compound 52 inhibits TPO mRNA expression in primary cultures of human thyrocytes from Donor 2 stimulated by bTSH or GD sera. Thyrocytes were incubated with bTSH (1.8 nM) or a 1:50 dilution of Graves' disease (GD) sera and 10 μM of compound 52 for 24 hours. Cells receiving 10 μM compound 52 were pre-incubated for 1 hour with the same concentration of compound 52 prior to the 24 hours incubation with bTSH. Data are presented as mean±SEM of two independent experiments.

FIG. 6 illustrates that intracellular cAMP accumulation in HEK-EM 293 cells stably expressing TSHR was determined in response to a 1:50 dilution of sera from patients with Graves' disease (GD) or the EC₅₀ concentration of bTSH (1.8 nM) in the presence or absence of compound 52. Serum from a patient with multinodular goiter was used as a control. Data are presented as mean±SEM of two independent experiments.

FIG. 7 illustrates cAMP data for two additional compounds—compounds 52/2 and 52/3, which have antagonistic activity at TSHR. Data are presented as mean±SEM of two independent experiments.

### DETAILED DESCRIPTION

### I. Introduction

Disclosed herein are small molecule compounds that can be used to modulate hormone receptors, such as seven transmembrane-spanning receptors. Because the seven-transmembrane helices of such receptors exhibit a high degree of homology it currently is believed, without limitation to any particular theory, that the disclosed compounds are useful for modulating many such receptors. Of particular interest is the modulation of the seven transmembrane-spanning receptors for luteinizing hormone/choriogonadotropin (LH/CG), follicle-stimulating hormone (FSH) and thyroid-stimulating hormone (TSH) which are heterodimeric glycoprotein hormones that regulate reproduction and thyroid homeostasis.

The TSH receptor regulates function of the thyroid gland and is important in several diseases. At present, recombinant human TSH (rhTSH, Thyrogen™) is an activator (agonist) of the TSH receptor that is used in the diagnosis and treatment of patients with thyroid cancer. In patients with hyperthyroidism (an “overactive thyroid”), the thyroid is overstimulated by antibodies (autoimmune hyperthyroidism or Graves's disease) or within a tumor (“toxic adenoma”) via the TSH receptor. An antagonist (inverse agonist) would inhibit the overstimulated thyroid and could be used to treat these forms of hyperthyroidism. Disclosed herein are low molecular weight compounds that bind to the TSH receptor and either activate it, like rhTSH, or down regulate it. Exemplary compounds may be used in methods of activating or down regulating the TSH receptor, according to the disclosed activity of the compound. Hence compounds that activate the TSH receptor can be used as receptor agonists, and compounds that inhibit the action of the TSH receptor can be used as antagonists.

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Variables such as R¹, R², R³, R⁴, R⁵, R⁶, R⁷, R⁸, R⁹, n, X and Y, used throughout the disclosure are the same variables as previously defined unless stated to the contrary.

“Optional” or “optionally” means that the subsequently described event or circumstance can but need not occur, and that the description includes instances where said event or circumstance occurs and instances where it does not.

“Derivative” refers to a compound or portion of a compound that is derived from or is theoretically derivable from a parent compound.

The term “subject” includes both human and veterinary subjects.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease or condition, for example, in a subject who is at risk for a disease such as a hormone receptor mediated disorder, particularly a thyroid disorder, such as a hyperthyroid or hypothyroid disorder. A “prophylactic” treatment is a treatment administered to a subject who does not exhibit signs of a disease or exhibits only early signs for the purpose of decreasing the risk of developing pathology. By the term “coadminister” is meant that each of at least two compounds be administered during a time frame wherein the respective periods of biological activity overlap. Thus, the term includes sequential as well as coextensive administration of two or more drug compounds.

The terms “pharmaceutically acceptable salt” or “pharmacologically acceptable salt” refers to salts prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Saturated or unsaturated” includes substituents saturated with hydrogens, substituents completely unsaturated with hydrogens and substituents partially saturated with hydrogens.

The term “acyl” refers group of the formula RC(O)— wherein R is an organic group.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkenyl” refers to a hydrocarbon group of 2 to 24 carbon atoms and structural formula containing at least one carbon-carbon double bond.

The term “alkynyl” refers to a hydrocarbon group of 2 to 24 carbon atoms and a structural formula containing at least one carbon-carbon triple bond.

The terms “halogenated alkyl” or “haloalkyl group” refer to an alkyl group as defined above with one or more hydrogen atoms present on these groups substituted with a halogen (F, Cl, Br, I).

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aliphatic” is defined as including alkyl, alkenyl, alkynyl, halogenated alkyl and cycloalkyl groups as described above. A “lower aliphatic” group is a branched or unbranched aliphatic group having from 1 to 10 carbon atoms.

“Alkoxycarbonyl” refers to an alkoxy substituted carbonyl radical, —C(O)OR, wherein R represents an optionally substituted alkyl, aryl, aralkyl, cycloalkyl, cycloalkylalkyl or similar moiety.

“Aminocarbonyl” alone or in combination, means an amino substituted carbonyl (carbamoyl) radical, wherein the amino radical may optionally be mono- or di-substituted, such as with alkyl, aryl, aralkyl, cycloalkyl, cycloalkylalkyl, alkanoyl, alkoxycarbonyl, aralkoxycarbonyl and the like.

The term “aryl” refers to any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted. The term “alkyl amino” refers to alkyl groups as defined above where at least one hydrogen atom is replaced with an amino group.

“Carbonyl” refers to a radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

“Carboxyl” refers to a —COOH radical. Substituted carboxyl refers to —COOR where R is aliphatic, heteroaliphatic, alkyl, heteroalkyl, or a carboxylic acid or ester.

The term “hydroxyl” is represented by the formula —OH. The term “alkoxy group” is represented by the formula —OR, where R can be an alkyl group, optionally substituted with an alkenyl, alkynyl, aryl, aralkyl, cycloalkyl, halogenated alkyl, or heterocycloalkyl group as described above.

The term “hydroxyaliphatic” refers to “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

The term “amine” or “amino” refers to a group of the formula —NRR′, where R and R′ can be, independently, hydrogen or an alkyl, alkenyl, alkynyl, aryl, aralkyl, cycloalkyl, halogenated alkyl, or heterocycloalkyl group described above.

The term “amide group” is represented by the formula —C(O)NRR′, where R and R′ independently can be a hydrogen, alkyl, alkenyl, alkynyl, aryl, aralkyl, cycloalkyl, halogenated alkyl, or heterocycloalkyl group described above.

The term “aralkyl” refers to an aryl group having an alkyl group, as defined above, attached to the aryl group. An example of an aralkyl group is a benzyl group.

Optionally substituted groups, such as “optionally substituted alkyl,” refers to groups, such as an alkyl group, that when substituted, have from 1-5 substituents, typically 1, 2 or 3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, halogen, optionally substituted heteroaryl, optionally substituted heterocyclyl, hydroxy, sulfonyl, thiol and thioalkoxy. In particular, optionally substituted alkyl groups include, by way of example, haloalkyl groups, such as fluoroalkyl groups, including, without limitation, trifluoromethyl groups.

Prodrugs of the disclosed hormone modulating compounds also are contemplated herein. A prodrug is an active or inactive compound that is modified chemically through in vivo physiological action, such as hydrolysis, metabolism and the like, into an active compound following administration of the prodrug to a subject. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985).

Pharmaceutically acceptable prodrugs refer to compounds that are metabolized, for example, hydrolyzed or oxidized, in the subject to form an antiviral compound of the present disclosure. Typical examples of prodrugs include compounds that have one or more biologically labile protecting groups on or otherwise blocking a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. In general the prodrug compounds disclosed herein possess hormone receptor modulating activity and/or are metabolized or otherwise processed in vivo to form a compound that exhibits such activity.

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently claimed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compound also are contemplated. A variety of suitable protecting groups for use with the disclosed compounds are disclosed in Greene and Wuts Protective Groups in Organic Synthesis; 3rd Ed.; John Wiley & Sons, New York, 1999.

In general, protecting groups are removed under conditions which will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts Protective Groups in Organic Synthesis; 3rd Ed.; John Wiley & Sons, New York, 1999.

When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed hormone receptor modulating compounds include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

It is understood that substituents and substitution patterns of the compounds described herein can be selected by one of ordinary skill in the art to provide compounds that are chemically stable and that can be readily synthesized by techniques known in the art and further by the methods set forth in this disclosure. Reference will now be made in detail to the presently preferred compounds.

### II. Hormone Receptor Modulating Compounds

Certain embodiments of the disclosed hormone receptor modulating compounds are represented by the formula

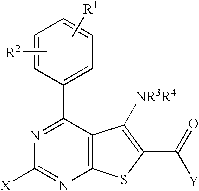

wherein X is —S(O)ₙR⁵;

n is 0, 1 or 2;

Y is —OR⁶ or —NR⁷R⁸

R¹ and R² independently are selected from optionally substituted lower aliphatic, alkoxy, aralkyl, halogen, hydrogen and —OR⁵, wherein R⁵ is selected from lower alkyl, hydrogen, aralkyl, acyl, alkoxycarbonyl and aminocarbonyl;

R³ and R⁴ independently are selected from acyl, alkoxycarbonyl, aminocarbonyl, aralkyl, hydrogen, lower alkyl and cycloalkyl;

R⁵ is selected from lower alkyl, aralkyl, cycloalkyl and haloalkyl;

R⁶ is selected from hydrogen, lower alkyl and aralkyl; and

R⁷ and R⁸ independently are selected from hydrogen, lower alkyl, aralkyl and cycloalkyl.

In one aspect such compounds have the formula

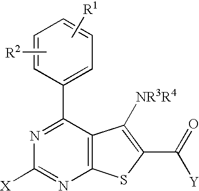

wherein X is —S(O)ₙR⁵;

n is 0, 1 or 2;

Y is —OR⁶ or —NR⁷R⁸

R¹ and R² independently are selected from optionally substituted lower aliphatic, alkoxy, aralkyl, halogen, H and —OR⁵, wherein R⁵ is selected from lower alkyl, H, aralkyl, acyl, alkoxycarbonyl and aminocarbonyl;

R³ and R⁴ independently are selected from acyl, alkoxycarbonyl, aminocarbonyl, aralkyl, H, lower alkyl and cycloalkyl;

R⁵ is selected from lower alkyl, aralkyl, cycloalkyl and haloalkyl;

R⁶ is selected from H, lower alkyl and aralkyl;

R⁷ and R⁸ independently are selected from H, lower alkyl, aralkyl and cycloalkyl; with the proviso that when R¹ is methoxy, R² is not H.

In certain embodiments of the disclosed hormone receptor modulating compounds, Y forms, together with the carbonyl moiety to which it is bound, an amide group. Such compounds can be represented by the formula

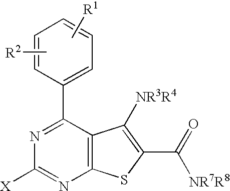

In certain disclosed compounds R⁷ and R⁸ independently are selected from hydrogen, lower alkyl, aralkyl and cycloalkyl. In certain examples of such compounds at least one of R⁷ and R⁸ is hydrogen. In particular embodiments, at least one of R⁷ and R⁸ is a sterically bulky substituent. Such sterically bulky substituents are known to those of ordinary skill in the art of organic chemistry and include alkyl groups, such as, without limitation, tert-butyl, iso-butyl, neopentyl, adamantyl and the like.

In certain embodiments, the disclosed compounds are represented by the formula

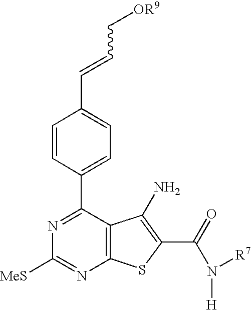

wherein R⁹ is selected from acyl, alkoxycarbonyl, aminocarbonyl, aralkyl, H, lower alkyl and cycloalkyl. With reference to the formula presented above, such compounds can be provided as single isomer or alternatively as mixtures of E and Z isomers. The E compounds, which are believed to be particularly effective antagonists of the TSH receptor can be represented by the formula

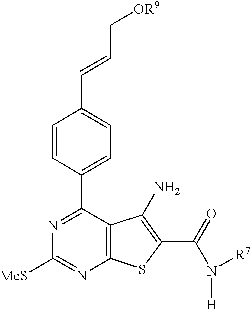

In other embodiments, there are provided compounds of the structure:

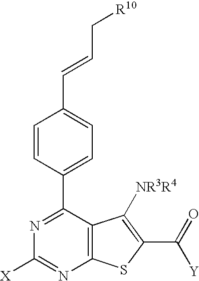

wherein R¹⁰ is —S(O)ₙR⁵ and R⁵ is lower alkyl (e.g., methyl) and n is 1 or 2; X is —S(O)ₙR⁵ and R⁵ is lower alkyl (e.g., methyl) and n is 1 or 2; and Y is —NR⁷R⁸. In certain embodiments, R⁷ and R⁸ are each independently H or lower alkyl, and R³ and R⁴ are each independently H or lower alkyl.

With reference to Table 1, exemplary disclosed compounds were evaluated against human TSH receptor and human LHCG receptor that were stably expressed in HEK 293 EM cells as previously described by Libert et al. (Biochem. Biophys. Res. Commun. 1989, 165, 1250-1255); and by Schulz et al. (Mol. Endocrinol. 1999, 13, 181-190). Cell surface expression of TSH receptor and LHCG receptor were determined via FACS analysis (Kleinau, G.; Jäschke, H.; Neumann, S.; Lättig, S.; Paschke, R.; Krause, G. J. Biol. Chem. 2004, 279, 51590-51600). Agonism of compounds 3-20 were determined via measurement of intracellular cyclic AMP accumulation. Certain embodiments of the disclosed hormone receptor modulating compounds exhibit advantageous receptor selectivity. For example, certain compound preferentially interact with the certain compounds disclosed herein exerted no discernible effect on the FSH receptor.

TABLE 1
Pharmacological characterization of selected hormone receptor modulating compounds at TSHR and LHCGR
stably expressed in HEK EM 293 cells

| | | | | | | | % Max. Resp. @ | | % Max. Resp. @ |
|---|---|---|---|---|---|---|---|---|---|
| | | | | | | EC₅₀ (LHCGR) in | LHCGR | EC₅₀ (TSHR) in | TSHR |
| Analogue # | X | R¹ | R² | R⁷ | R⁸ | μM [95% C.I.] | in μM | μm [95% C.I.] | in μM |
| 3 | N | OMe | H | tBu | H | 0.3 [0.2-0.5] | 45.8 ± 5.9 | 6.5 [4.9-8.5] | 23.4 ± 3.6 |
| 4 | O | OMe | H | Et | H | n.d. | 4.2 ± 2.2 | n.d. | 1.5 ± 0.2 |
| 5 | O | OMe | H | tBu | H | 1.1 [0.8-1.5] | 23.8 ± 3.3 | 11.9** | >30.3* |
| 6 | N | OMe | H | Et | H | n.d. | 26.9 ± 4.8 | n.d. | 2.3 ± 0.4 |
| 7 | N | OMe | H | tBu | Me | 0.8 [0.6-1.2] | 47.8 ± 2.8 | n.d. | 3.6 ± 1.9 |
| 8 | N | OMe | H | NH₂ | H | n.d. | 8.5 ± 3.6 | n.d. | 6.1 ± 0.8 |
| 9 | N | OMe | H | N(Me)₂ | H | n.d. | 11.0 ± 1.5 | n.d. | 4.0 ± 0.1 |
| 10 | N | OMe | H | NH(tBu) | H | n.d. | 6.0 ± 3.1 | n.d. | 6.4 ± 0.6 |
| 11 | N | OMe | H | NH(Boc) | H | n.d. | 2.4 ± 0.5 | n.d. | 1.9 ± 0.4 |
| 12 | N | OMe | H | 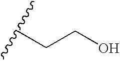 | H | n.d. | 20.5 ± 2.7 | n.d. | 2.9 ± 0.5 |
| 13 | N | OMe | H | 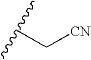 | H | n.d. | 20.3 ± 2.1 | n.d. | 3.6 ± 0.6 |
| 14 | N | OMe | H | 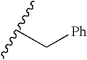 | H | n.d. | 7.8 ± 2.7 | n.d. | 3.0 ± 1.2 |
| 15 | N | OMe | H | 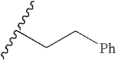 | H | n.d. | 25.6 ± 5.4 | n.d. | 4.3 ± 0.4 |
| 16 | N | OMe | OMe | tBu | H | 0.8 [0.7-1.0] | 50.1 ± 3.6 | n.d. | 3.0 ± 0.9 |
| 17 | N | OMe | F | tBu | H | 1.5 [1.0-2.1] | 46.3 ± 6.6 | 11.5** | >24.0* |
| 18 | N | F | H | tBu | H | 1.2 [0.8-1.6] | 51.1 ± 5.2 | n.d. | 8.1 ± 1.7 |
| 19 | N | OH | H | tBu | H | 1.9 [1.1-3.4] | 63.9 ± 14.2 | n.d. | 11.2 ± 1.0 |

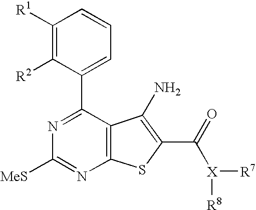

Agnostic activity of compounds was determined via measurement of intracellular cyclic AMP. The efficacy
(maximum response) is expressed as % of maximum response of LHCGR or TSHR to LH (1000 ng/ml)
or TSH (100 mU/ml), respectively. EC₅₀ values and 95% confidence intervals (C.I.) were obtained from
dose response curves (0-100 μM compound) using the GraphPad Prism 4.0 software.
Confidence intervals were not calculated in dose response curves that did not reach an abvious plateau.
n.d. = not determined
*Estimated maximum response at 100 μM compound
**Estimated EC₅₀ (dose response curve revealed no plateau)

The specification and claims contain listing of species using the language “selected from the group consisting of . . . and . . . ” and “selected from the group consisting of . . . or . . . ” (sometimes referred to as Markush groups). When this language is used in this application, unless otherwise stated it is meant to include the group as a whole, any single members thereof, or any subgroups thereof. The use of this language is merely for shorthand purposes and is not meant in any way to limit the removal of individual elements or subgroups as needed.

Pharmaceutical compositions that comprise N-tert-butyl-5-amino-4-(4-((E)-but-1-enyl)phenyl)-2-(methylthio)thieno[2,3-d]pyrimidine-6-carboxamide are particularly useful, for example, to inhibit TSH receptor activation. For example, this compound has been demonstrated to inhibit the activation of TSH receptor by antibodies (IgG) from Graves' disease sera.

### III. Synthesis

With reference to Scheme 1, the synthesis disclosed hormone receptor modulating compounds was accomplished in a similar manner to that described by van Boeckel and coworkers (van Straten, N. C. R. Schoonus-Gerritsma, G. G.; van Someren, R. G.; Draaijer, J.; Adang, A. E. P.; Timmers, C. M.; Hanseen, R. G. J. M.; van Boeckel, C. A. A. Chem. Bio. Chem. 2002, 10, 1023). With continued reference to Scheme 1, a modified Biginelli condensation (step i) afforded the substituted pyrimidone scaffold.

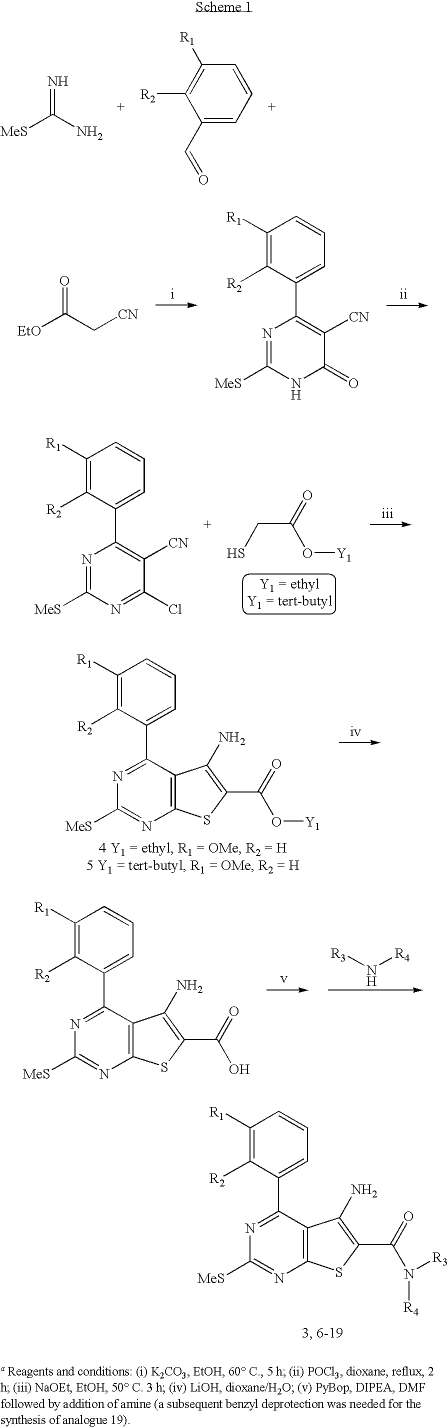

Numerous aldehydes were tolerated within this system, including highly electron withdrawn (i.e. polyfluoro and nitro) and electron rich (polymethoxy and hydroxyl) aromatic ring systems. Treatment with POCl₃ afforded the 4-chloro-substituted pyrimidines in quantitative yields and substitution with either ethyl-2-mercaptoacetate or tert-butyl-2-mercaptoacetate afforded several thienopyrimidines, including biochemically relevant compounds 4 and 5. Saponification of the ethyl esters with lithium hydroxide in a dioxane/water mixture provided the thienopyrimidine acids and PyBOP catalyzed amide couplings with several amines provided Org 41841 (3) and compounds 6-19.

Initial docking experiments suggested a potential hydrogen bond between the amine functionality of 3 and E3.37 in transmembrane helix 3 of both TSH receptor and LHCG receptor. To fully examine this we chose to eliminate this potential interaction via two distinct experimental means. Using the small molecule as a point of manipulation, the removal of the aromatic amine or the protection of the aromatic amine via dimethylation would accomplish the exclusion of H-bond donation capability. Unfortunately, all attempts to deaminate the Org 41841 structure were unsuccessful. However, direct treatment with methyl iodide in basic acetonitrile afforded the dimethylamine compound (20) along with the monomethylated analogue and the concomitant dimethyl amine-methyl amide addition. Purification via HPLC was performed prior to biological evaluation of 20.

### IV. Compositions, Administration and Use of the Disclosed Compounds

Another aspect of the disclosure includes pharmaceutical compositions prepared for administration to a subject and which include a therapeutically or diagnostically effective amount of one or more of the currently disclosed compounds. The therapeutically effective amount of a disclosed compound will depend on the route of administration, the species of subject and the physical characteristics of the subject being treated or evaluated. Specific factors that can be taken into account include disease severity and stage, weight, diet and concurrent medications. The relationship of these factors to determining a therapeutically or spectroscopically effective amount of the disclosed compounds is understood by those of skill in the art. In general, however, a suitable dose for consideration will be in the range of analogous hormone receptor agonists and antagonists, taking into account differences in potency observed in vitro testing, generally from about 0.1 to 400 mg per kilogram body weight of the subject per dose, such as in a range between about 0.1 mg and about 250 mg/kg/dose in increments of 0.5 mg/kg/dose such as 2.5 mg/kg/dose, 3.0 mg/kg/dose, 3.5 mg/kg/dose, etc), typically in the range 0.5 to 50 mg per kilogram body weight per dose and most usually in the range 1 to 300 mg per kilogram body weight per dose. The exact dosage and regimen for administration of the presently disclosed compounds will be dependent on the therapeutic effect sought (for example, thyroid modulation, infertility treatment, contraception) and may vary with the particular compound and individual subject to whom the compound is administered. The desired dose may be presented as one dose or as multiple subdoses administered at appropriate intervals throughout the day, or, in case of female recipients, as doses to be administered at appropriate daily intervals throughout the menstrual cycle. The dosage as well as the regimen of administration may differ between a female and a male recipient. In case of in vitro or ex vivo applications, such as in vitro fertilization applications, the compounds of the inventions are to be used in the incubation media in a concentration of approximately 0.01-5 μg/mL.

Pharmaceutical compositions for administration to a subject can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like. Pharmaceutical formulations can include additional components, such as carriers. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the disclosed compounds.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Pharmaceutical compositions suitable for oral administration may be presented as discrete dosage units such as pills, tablets or capsules, or as a powder or granules, or as a solution or suspension. The active ingredient may also be presented as a bolus or paste. The compositions can further be processed into a suppository or enema for rectal administration.

For parenteral administration, suitable compositions include aqueous and non-aqueous sterile injection. The compositions may be presented in unit-dose or multi-dose containers, for example sealed vials and ampoules, and may be stored in a freeze-dried (lyophilized) condition requiring only the addition of sterile liquid carrier, for example, water prior to use.

Compositions, or formulations, suitable for administration by nasal inhalation include fine dusts or mists which may be generated by means of metered dose pressurized aerosols or nebulizers.

The disclosed compounds also can be administered in the form of implantable pharmaceutical devices, consisting of a core of active material, encased by a release rate-regulating membrane. Such implants are to be applied subcutaneously or locally, and will release the active ingredient at an approximately constant rate over relatively large periods of time, for instance from weeks to years. Methods for the preparation of implantable pharmaceutical devices as such are known in the art, for example as described in European Patent 6,303,306 (AKZO N.V.).

The disclosed hormone receptor modulators can be administered to any subject in need thereof. Suitable compounds for treating subjects can be selected in part based on the condition to be treated. For example, certain compounds are TSH receptor antagonists. Such antagonist compounds may be used to treat disorders of hyperthyroidism, such as Graves' disease.

Follicle stimulating hormone currently is in clinical use for treating infertility. The disclosed FSH receptor agonists can be used to replace follicle stimulating hormone as infertility therapeutics. Similarly, compounds disclosed herein that have luteinizing hormone (LH) receptor activating activity can be used in fertility regulating therapies. For example, certain LH receptor activating compounds disclosed herein can be used for the same clinical purposes as native luteinizing hormone, with the advantage that the disclosed compounds display superior stability properties and thus can be administered differently. Thus, examples of the disclosed low molecular weight ligands of LHCG receptor and FSH receptor can be used as therapeutics for infertility treatment or oral contraception. It is noteworthy that in vivo efficacy of Organon lead compound Org41841 (N-tert-butyl-5-amino-4-(3-methoxyphenyl)-2-(methylthio)thieno[2,3-d]pyrimidine-6-carboxamide) for LHCG receptor was demonstrated in an ovulation induction model supporting the pharmacological utility of the synthetic ligands disclosed herein (van Straten, N. C., Schoonus-Gerritsma, G. G., van Someren, R. G., Draaijer, J., Adang, A. E., Timmers, C. M., Hanssen, R. G., and van Boeckel, C. A. (2002) Chembiochem. 3, 1023-1026). Similarly, the low molecular weight antagonists of TSH receptor have therapeutic application in treating TSH receptor-mediated hyperthyroidism and agonists might replace injected recombinant human TSH (rhTSH, Thyrogen™) in diagnostic screening for thyroid cancer.

### EXAMPLES

The following examples are intended to be illustrative rather than limiting.

### General Methods

¹H NMR data was recorded on a Varian Gemini 300 MHz. Spectra were recorded in d₆-DMSO, d₄-CD₃OD, and D₂O and were referenced to the residual solvent peak at 2.50, 3.31 and 4.79 ppm, respectively. Reverse-phase (C18) HPLC was carried out using an Agilent HPLC with a Zorbax™ SP-C18 semi-prep column. High-resolution mass spectroscopy measurements were performed on a Micromass/Waters LCT Premier Electrospray TOF mass spectrometer.

### General Synthetic Procedures

The following general procedures were used to synthesize compounds having different but analogous structures. One of skill in the art will recognize how to modify these general procedures if necessary to accomplish the desired transformations.

5-carbonitrile-1,6-dihydro-2-(methylthio)-6-oxo-4-(substituted phenyl)pyrimidines. To a solution of S-methylisothiourea (1 equiv), the appropriately substituted benzaldehyde (2 equiv) and ethyl cyanoacetate (2 equiv) in ethanol was added K₂CO₃ (2 equiv). The reaction mixture was heated to 60° C. for 5 h and filtered upon cooling to obtain products. Purification by flash chromatography (using EtOAc:hexane 1:1) provided the final products as off white solids in 30-50% yields.

5-carbonitrile-4-chloro-2-(methylthio)-6-(3-substituted phenyl)pyrimidines. To a mixture of the oxopyrimidines in dioxane was added POCl₃ (excess) in dioxane. The reaction was heated to reflux for 3 h and the solvent was removed by reduced pressure. Saturated NaHCO₃ was added to the resulting brown solids and the reaction mixtures were extracted with CH₂Cl₂ (3×100 mL). The organic layers were combined, dried over Na₂SO₄, and the solvent was removed under reduced pressure. Purification by silica plug filtration (using EtOAc:hexane 1:1) provided the final products as white crystalline solids in 80-90% yields.

ethyl-5-amino-2-(methylthio)-4-(substituted phenyl)thieno[2,3-d]pyrimidine-6-carboxylates. To a solution of the appropriate pyrimidine (1 equiv) and ethyl-2-mercaptoacetate- or -tert-butyl-2-mercaptoacetate (1.1 equiv) in ethanol was added sodium (0.910 equiv) in ethanol. The yellow reaction mixture was heated to 50° C. for 3 h, cooled and the ethanol removed under reduced pressure. The yellow solids were dissolved in CH₂Cl₂ (50 mL), washed with DI water (3×25 mL), the organic layer was dried over Na₂SO₄, and the solvent removed under reduced pressure. Purification by flash chromatography (using EtOAc:hexane 1:1) provided the final products as yellow solids in 70-90% yields.

N-tert-butyl-5-amino-2-(methylthio)-4-(substituted phenyl)thieno[2,3-d]pyrimidine-6-carboxamides. To a solution of the appropriate ethyl ester (I equiv) in a dioxane and water mixture was added lithium hydroxide (2 equiv). The reaction mixture was heated to 50° C. for 3 h, cooled and the solvent removed under reduced pressure. The crude acid was used without further purification. The yellow solids were dissolved in a minimal amount of DMF, followed by the addition of PyBOP (3 equiv), DIPEA (5.5 equiv) and tert-butylamine (3 equiv), respectively. Purification by flash chromatography (using EtOAc:hexane 2:1) provided the final products as yellow solids in 50-90% yields.

The following examples describe the purification and characterization of disclosed hormone receptor modulating compounds and intermediates and analogs thereof.

N-tert-butyl-5-amino-4-(3-methoxyphenyl)-2-(methylthio)thieno[2,3-d]pyrimidine-6-carboxamide (3). Analysis by C₈ reversed phase LCMS using a linear gradient of H₂O with increasing amounts of CH₃CN (0→17 min, 30%→70% CH₃CN at a flow rate of 1 mL/min, t_{R} 13.5 min) found greater than 99% purity by peak integration. ¹H NMR (CDCl₃) δ 1.45 (s, 9H), 2.64 (s, 3H), 3.86 (s, 3H), 5.99 (br. s, 2H), 7.07-7.26 (m, 3H), 7.41-7.47 (m, 1H); mass spectrometry (TOF); m/z=403.1262 (M+H⁺) (theoretical 403.1257).

ethyl-5-amino-4-(3-methoxyphenyl)-2-(methylthio)thieno[2,3-d]pyrimidine-6-carboxylate (4). Analysis by C₈ reversed phase LCMS using a linear gradient of H₂O with increasing amounts of CH₃CN (0→15 min, 30%→90% CH₃CN at a flow rate of 1 mL/min, t_{R} 12.5 min) found greater than 92% purity by peak integration. ¹H NMR (d₆-DMSO) δ 1.37 (t, J=7.2 Hz, 3H), 2.69 (s, 3H), 3.92 (s, 3H), 4.35 (q, J=7.2 Hz, 2H), 6.15 (br. s, 2H), 7.27-7.31 (m, 3H), 7.59-7.64 (m, 1H); mass spectrometry (TOF); m/z=376.0790 (M+H⁺) (theoretical 376.0784).

tert-butyl-5-amino-4-(3-methoxyphenyl)-2-(methylthio)thieno[2,3-d]pyrimidine-6-carboxylate (5). Analysis by C₈ reversed phase LCMS using a linear gradient of H₂O with increasing amounts of CH₃CN (0→15 min, 30%→90% CH₃CN at a flow rate of 1 mL/min, t_{R} 13.2 min) found greater than 98% purity by peak integration. ¹H NMR (CDCl₃) δ 1.57 (s, 9H), 2.64 (s, 3H), 3.86 (s, 3H), 5.78 (br. s, 2H), 7.08-7.16 (m, 3H), 7.42-7.47 (m, 1H); mass spectrometry (TOF); m/z=404.1097 (M+H⁺) (theoretical 404.1103).

5-amino-N-(ethyl)-4-(3-methoxyphenyl)-2-(methylthio)thieno[2,3-d]pyrimidine-6-carboxamide (6). Analysis by C₈ reversed phase LCMS using a linear gradient of H₂O with increasing amounts of CH₃CN (0→18 min, 40%→80% CH₃CN at a flow rate of 1 mL/min, t_{R} 9.8 min) found greater than 99% purity by peak integration. ¹H NMR (d₆-DMSO) δ 1.08 (t, J=7.2 Hz, 3H), 2.59 (s, 3H), 3.22 (p, J=7.2 Hz, 2H), 3.82 (s, 3H), 5.75 (s, 1H), 6.10 (br. s, 2H), 7.15-7.19 (m, 2H), 7.50 (t, J=8.0 Hz, 1H), 7.87 (t, J=8.0 Hz, 1H); mass spectrometry (TOF); m/z=375.0944 (M+H⁺) (theoretical 375.0949).

N-tert-butyl-5-amino-4-(3-methoxyphenyl)-N-methyl-2-(methylthio)thieno[2,3-d]pyrimidine-6-carboxamide (7). Analysis by C₈ reversed phase LCMS using a linear gradient of H₂O with increasing amounts of CH₃CN (0→18 min, 40%→80% CH₃CN at a flow rate of 1 mL/min, t_{R} 14.6 min) found greater than 95% purity by peak integration. ¹H NMR (d₆-DMSO) δ 1.36 (s, 9H), 2.58 (s, 3H), 3.00 (s, 3H), 3.82 (s, 3H), 5.22 (br. s, 2H), 7.17-7.20 (m, 3H), 7.51 (t, J=8 Hz, 1H); mass spectrometry (TOF); m/z=417.1413 (M+H⁺) (theoretical 417.1419).

5-amino-4-(3-methoxyphenyl)-2-(methylthio)thieno[2,3-d]pyrimidine-6-carbohydrazide (8). Analysis by C₈ reversed phase LCMS using a linear gradient of H₂O with increasing amounts of CH₃CN (0→18 min, 40%→80% CH₃CN at a flow rate of 1 mL/min, t_{R} 13.7 min) found greater than 92% purity by peak integration. ¹H NMR (d₆-DMSO) δ 2.59 (s, 3H), 3.82 (s, 3H), 6.18 (br. s, 2H), 7.17-7.20 (m, 3H), 7.50 (t, J=8.7 Hz, 1H), 9.20 (br. s, 1H); mass spectrometry (TOF); m/z=362.074 (M+H⁺) (theoretical 362.0745).

5-amino-4-(3-methoxyphenyl)-N′N′-dimethyl-2-(methylthio)thieno[2,3-d]pyrimidine-6-carbohydrazide (9). Analysis by C₈ reversed phase LCMS using a linear gradient of 0.1% TFA in H₂O with increasing amounts of CH₃CN (0→18 min, 30%→80% CH₃CN at a flow rate of 1 mL/min, t_{R} 8.7 min) found greater than 93% purity by peak integration. ¹H NMR (d₆-DMSO) δ 2.55 (s, 6H), 2.58 (s, 3H), 3.82 (s, 3H), 6.45 (br. s, 2H), 7.16-7.18 (m, 3H), 7.50 (t, J=8.7 Hz, 1H), 8.72 (s, 1H); mass spectrometry (TOF); m/z=390.1053 (M+H⁺) (theoretical 390.1058).

N′-tert-butyl-5-amino-4-(3-methoxyphenyl)-2-(methylthio)thieno[2,3-d]pyrimidine-6-carbohydrazide (10). Analysis by C₈ reversed phase LCMS using a linear gradient of H₂O with increasing amounts of CH₃CN (0→10 min, 25%→90% CH₃CN, 10→15 min, 90%→25% CH₃CN at a flow rate of 1 mL/min, t_{R} 12.0 min) found greater than 95% purity by peak integration. ¹H NMR (d₆-DMSO) δ 1.08 (s, 9H), 2.59 (s, 3H), 3.82 (s, 3H), 4.93 (s, 1H), 6.45 (br. s, 2H), 7.16-7.18 (m, 3H), 7.50 (t, J=8 Hz, 1H), 8.53 (s, 1H); mass spectrometry (TOF); m/z=418.1366 (M+H⁺) (theoretical 418.1371).

N′-Boc-5-amino-4-(3-methoxyphenyl)-2-(methylthio)thieno[2,3-d]pyrimidine-6-carbohydrazide (11). Analysis by C₈ reversed phase LCMS using a linear gradient of H₂O with increasing amounts of CH₃CN (0→10 min, 25%→90% CH₃CN, 10→15 min, 90%→25% CH₃CN at a flow rate of 1 mL/min, t_{R} 11.0 min) found greater than 97% purity by peak integration. ¹H NMR (d₆-DMSO) δ 1.08 (s, 9H), 2.59 (s, 3H), 3.82 (s, 3H), 4.93 (s, 1H), 6.45 (br. s, 2H), 7.16-7.18 (m, 3H), 7.50 (t, J=8 Hz, 1H), 8.53 (s, 1H); mass spectrometry (TOF); m/z=462.1264 (M+H⁺) (theoretical 462.127).

5-amino-4-(3-methoxyphenyl)-N-(2-hydroxyethyl)-2-(methylthio)thieno[2,3-d]pyrimidine-6-carboxamide (12) Analysis by C₈ reversed phase LCMS using a linear gradient of H₂O with increasing amounts of CH₃CN (0→18 min, 30%→60% CH₃CN at a flow rate of 1 mL/min, t_{R} 7.4 min) found greater than 92% purity by peak integration. ¹H NMR (d₆-DMSO) δ 2.59 (s, 3H), 3.20-3.40 (m, 2H), 3.41-3.55 (m, 2H), 3.82 (s, 3H), 4.71 (m, 1H), 6.10 (br. s, 2H), 7.19 (br. s, 2H), 7.50 (m, 1H), 7.80 (m, 1H); mass spectrometry (TOF); m/z=391.0893 (M+H⁺) (theoretical 391.0899).

5-amino-N-(cyanomethyl)-4-(3-methoxyphenyl)-2-(methylthio)thieno[2,3-d]pyrimidine-6-carboxamide (13). Analysis by C₈ reversed phase LCMS using a linear gradient of H₂O with increasing amounts of CH₃CN (0→10 min, 25%→90% CH₃CN, 10→15 min, 90%→25% CH₃CN at a flow rate of 1 mL/min, t_{R} 10.5 min) found greater than 91% purity by peak integration. ¹H NMR (d₆-DMSO) δ 2.60 (s, 3H), 3.82 (s, 3H), 4.22 (d, J=5.4 Hz, 2H), 6.23 (br. s, 2H), 7.18-7.20 (m, 3H), 7.51 (t, J=8.1 Hz, 1H), 8.53 (t, J=5.4 Hz, 1H); mass spectrometry (TOF); m/z=386.074 (M+H⁺) (theoretical 386.0745).

5-amino-N-benzyl-4-(3-methoxyphenyl)-2-(methylthio)thieno[2,3-d]pyrimidine-6-carboxamide (14). Analysis by C₈ reversed phase LCMS using a linear gradient of H₂O with increasing amounts of CH₃CN (0→18 min, 40%→80% CH₃CN at a flow rate of 1 mL/min, t_{R} 12.6 min) found greater than 99% purity by peak integration. ¹H NMR (d₆-acetone) δ 2.61 (s, 3H), 3.89 (s, 3H), 4.55 (d, J=6 Hz, 2H), 6.29 (br. s, 2H), 7.18-7.37 (m, 8H), 7.49 (t, J=8.4 Hz, 1H), 7.64 (t, J=3 Hz, 1H); mass spectrometry (TOF); m/z=437.1100 (M+H⁺) (theoretical 437.1106).

5-amino-4-(3-methoxyphenyl)-2-(methylthio)-N-phenethylthieno[2,3-d]pyrimidine-6-carboxamide (15). Analysis by C₈ reversed phase LCMS using a linear gradient of H₂O with increasing amounts of CH₃CN (0→18 min, 40%→80% CH₃CN at a flow rate of 1 mL/min, t_{R} 14.7 min) found greater than 98% purity by peak integration. ¹H NMR (d₆-DMSO) δ 2.59 (s, 3H), 2.81 (t, J=7.2 Hz, 2H), 3.43 (q, J=8.4 Hz, 2H), 3.82 (s, 3H), 6.11 (br. s, 2H), 7.16-7.32 (m, 8H), 7.50 (t, J=7.8 Hz, 1H), 7.96 (t, J=3 Hz, 1H); mass spectrometry (TOF); m/z=451.1257 (M+H⁺) (theoretical 451.1262).

N-tert-butyl-5-amino-4-(2,3-dimethoxyphenyl)-2-(methylthio)thieno[2,3-d]pyrimidine-6-carboxamide (16). Analysis by C₈ reversed phase LCMS using a linear gradient of H₂O with increasing amounts of CH₃CN (0→16 min, 35%→95% CH₃CN at a flow rate of 1 mL/min, t_{R} 14.3 min) found greater than 93% purity by peak integration. ¹H NMR (CDCl₃) δ 1.45 (s, 9H), 2.66 (s, 3H), 3.76 (s, 3H), 3.95 (s, 3H), 5.77 (br. s, 2H), 6.91 (dd, J=1.3, 7.5 Hz, 1H), 7.21 (t, J=8.2 Hz, 1H); mass spectrometry (TOF); m/z=433.1363 (M+H⁺) (theoretical 433.1368).

N-tert-butyl-5-amino-4-(2-fluoro-3-methoxyphenyl)-2-(methylthio)thieno[2,3-d]pyrimidine-6-carboxamide (17). Analysis by C₈ reversed phase LCMS using a linear gradient of H₂O with increasing amounts of CH₃CN (0→15 min, 35%→90% CH₃CN at a flow rate of 1 mL/min, t_{R} 11.0 min) found greater than 92% purity by peak integration. ¹H NMR (CDCl₃) δ 1.44 (s, 9H), 2.63 (s, 3H), 3.95 (s, 3H), 5.79 (br. s, 2H), 6.98 (dt, J=7.5, 1.8 Hz, 1H), 7.15 (dt, J=8.1, 1.8 Hz, 1H), 7.24 (t, J=7.5 Hz, 1H); mass spectrometry (TOF); m/z=421.1163 (M+H⁺) (theoretical 421.1168).

N-tert-butyl-5-amino-4-(3-fluorophenyl)-2-(methylthio)thieno[2,3-d]pyrimidine-6-carboxamide (18). Analysis by C₈ reversed phase LCMS using a linear gradient of H₂O with increasing amounts of CH₃CN (0→15 min, 45%→90% CH₃CN at a flow rate of 1 mL/min, t_{R} 11.4 min) found greater than 92% purity by peak integration. ¹H NMR (CDCl₃) δ 1.54 (s, 9H), 2.68 (s, 3H), 7.19-7.50 (m, 4H); mass spectrometry (TOF); m/z=391.1072 (M+H⁺) (theoretical 391.1057).

N-tert-butyl-5-amino-4-(3-hydroxyphenyl)-2-(methylthio)thieno[2,3-d]pyrimidine-6-carboxamide (19). Analysis by C₈ reversed phase LCMS using a linear gradient of H₂O with increasing amounts of CH₃CN (0→10 min, 25%→90% CH₃CN at a flow rate of 1 mL/min, t_{R} 10.1 min) found greater than 92% purity by peak integration. ¹H NMR (CDCl₃) δ 1.45 (s, 9H), 2.64 (s, 3H), 5.98 (br. s, 2H), 7.02 (d, J=7.2 Hz, 2H), 7.12 (d, J=7.5 Hz, 1H) 7.39 (t, J=7.8 Hz, 1H); mass spectrometry (TOF); m/z=389.110 (M+H⁺) (theoretical 389.1106).

N-tert-butyl-5-(-dimethylamino)-4-(3-methoxyphenyl)-2-(methylthio)thieno[2,3-d]pyrimidine-6-carboxamide (20). Analysis by C₈ reversed phase LCMS using a linear gradient of H₂O with increasing amounts of CH₃CN (0→5 min, 50%→90% CH₃CN, 5→15 min, 90% CH₃CN at a flow rate of 1 mL/min, t_{R} 7.4 min) found greater than 93% purity by peak integration. ¹H NMR (CDCl₃) δ 1.43 (s, 9H), 2.37 (s, 6H), 2.63 (s, 3H), 3.84 (s, 3H), 7.03 (d, J=8.4 Hz, 1H), 7.09-7.11 (m, 2H), 7.38 (t, J=8.1 Hz, 1H), 7.48 (br s, 1H); mass spectrometry (TOF); m/z=431.1553 (M+H⁺) (theoretical 431.1575).

### Confirmation of Structure and Purity

The structural characterization and purity of the above listed compounds were confirmed as follows for:

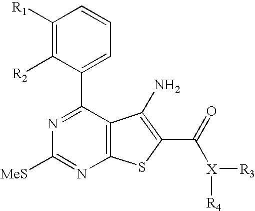

| | | | | | | | HPLC | | | | | |
|---|---|---|---|---|---|---|---|---|---|---|---|---|
| | | | | | | | Rt | | HPLC | | HRMS | HRMS |
| | | | | | | | (min) | HPLC | Rt | HPLC | theo. | found |
| # | X | R1 | R2 | R3 | R4 | R5 | a | purity | (min) b | purity | (m/z) | (m/z) |
| 3 | N | OMe | H | tBu | H | H | 7.336 | 98% | 11.927 | 98% | 403.1257 | 403.1262 |
| 4 | O | OMe | H | Et | H | H | 7.494 | 96% | 12.201 | 85% | 376.0784 | 376.0790 |
| 5 | O | OMe | H | tBu | H | H | 8.991 | 99% | 14.695 | 98% | 404.1097 | 404.1103 |
| 6 | N | OMe | H | Et | H | H | 5.735 | 99% | 9.698 | 99% | 379.0944 | 375.0949 |
| 7 | N | OMe | H | tBu | Me | H | 7.851 | 98% | 12.647 | 97% | 417.1413 | 417.1419 |
| 8 | N | OMe | H | NH₂ | H | H | 4.452 | 84% | 7.272 | 80% | 362.0740 | 362.0745 |
| 9 | N | OMe | H | N(Me)₂ | H | H | 5.734 | 95% | 9.639 | 90% | 390.1053 | 390.1058 |
| 10 | N | OMe | H | NH(tBu) | H | H | 6.272 | 99% | 10.484 | 98% | 418.1366 | 418.11371 |
| 11 | N | OMe | H | NH(Boc) | H | H | 5.385 | 99% | 9.375 | 99% | 462.1264 | 462.1270 |
| 12 | N | OMe | H | EtOH | H | H | 4.257 | 98% | 7.064 | 97% | 391.0893 | 391.0899 |
| 13 | N | OMe | H | CH2CN | H | H | 5.107 | 85% | 8.762 | 81% | 386.0740 | 386.0745 |
| 14 | N | OMe | H | Bm | H | H | 6.483 | 98% | 10.876 | 99% | 437.1100 | 437.1106 |
| 15 | N | OMe | H | CH₂CH₂Ph | H | H | 6.821 | 99% | 11.301 | 99% | 451.1257 | 451.1262 |
| 16 | N | OMe | OMe | tBu | H | H | 6.899 | 97% | 11.354 | 96% | 433.1363 | 433.1368 |
| 17 | N | OMe | F | tBu | H | H | 6.798 | 93% | 11.247 | 95% | 421.1163 | 421.1168 |
| 18 | N | F | H | tBu | H | H | 7.315 | 98% | 11.919 | 98% | 391.1057 | 391.1072 |
| 19 | N | OH | H | tBu | H | H | 5.848 | 91% | 9.926 | 90% | 389.1100 | 389.1106 |
| 20 | N | OMe | H | tBu | H | Me | 7.39 | 95% | 12.76 | 91% | 431.1575 | 431.1553 |

a linear gradient of H₂O containing increasing amounts of CH3CN (0-5 min, linear gradient from 50%-95% CH3CN; 5-14.9 min, gradient maintained at 95% CH₃CN).
b linear gradient of H₂O containing increasing amounts of CH3CN (0-7 min, linear gradient from 30%-80% CH₃CN; 7-8 min, 80-90% CH3CN; 8-13 min, gradient maintained at 90%; 13-14 min, linear gradient 90%-30% CH3CN; 14-15 min, gradient maaintained at 30% CH3CN).

Tissue Culture and cAMP Assay

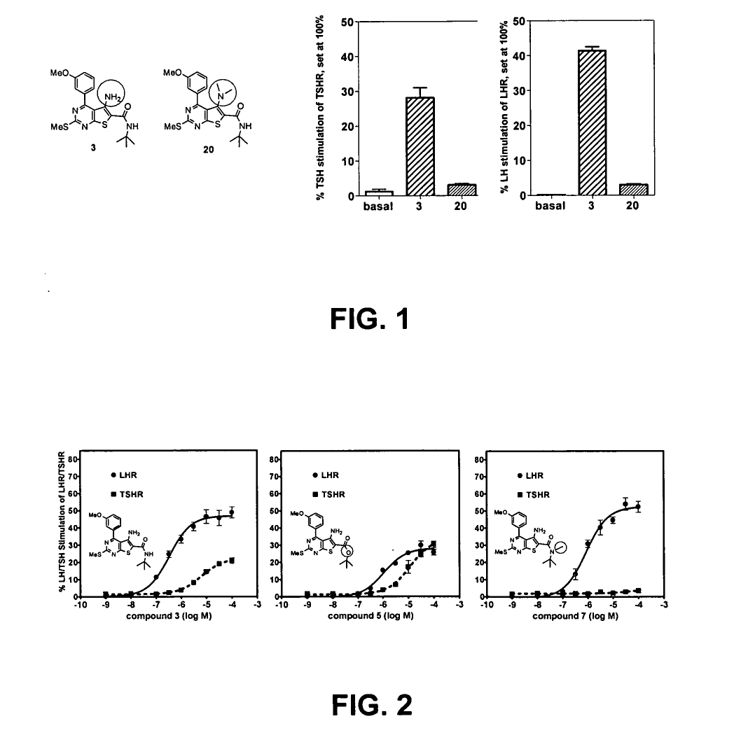

Cells were cultured for 48 h in 24-well plates before incubation for 1 h in serum-free DMEM containing 1 mM 3-isobutyl-1-methylxanthine (IBMX) (SIGMA) and bovine TSH (1.8 μM) (SIGMA) or human LH (1000 ng/ml) (Dr. A. Parlow, NIDDK National Hormone and Pituitary Program) or compounds 3-19 (0-100 μM) in a humidified 5% CO₂ incubator. Following aspiration of the medium after incubation with compounds, cells were lysed using lysis buffer 1 of the cAMP Biotrak Enzymeimmunoassay (EIA) System (Amersham Biosciences). The cAMP content of the cell lysate was determined using the manufacturer's protocol. The efficacy of receptor activation by small molecule modulators is expressed as % of maximum response of LHCG receptor or TSH receptor to LH or TSH, respectively. The potency (EC₅₀) was obtained from dose response curves (0-100 μM compound) by data analysis with GraphPad Prism 4 for Windows. With reference to FIG. 1, intracellular cAMP production was determined in response to 100 μM of each compound and is expressed as % of maximum response of TSHR/LHCGR to TSH (100 mU/ml)/LH (1000 ng/ml). The data are presented as mean±SEM of two independent experiments, each performed in duplicate.

To determine cell surface expression, cells were cultured after transfection for 48 h, harvested using 1 mM EDTA/1 mM EGTA in PBS and transferred to Falcon 2058 tubes. Cells were washed once with PBS containing 0.1% BSA and 0.1% NaN₃ (binding buffer), incubated for 1 h with a 1:200 dilution of mouse anti-human TSH receptor antibody (Serotec) in binding buffer, washed twice and incubated for 1 h in the dark with a 1:200 dilution of an Alexa Fluor 488-labeled F(ab′)₂ fragment of goat anti-mouse IgG (Molecular Probes) in binding buffer. Before FACS analysis (FACS Calibur, BD Biosciences), cells were washed twice and fixed with 1% paraformaldehyde. Receptor expression was estimated by fluorescence intensity and transfection efficiency was estimated from the percentage of fluorescent cells.

### Examples

### Compounds 52, 52/1, 52/2 and 52/3

Compound 52 has the following structure:

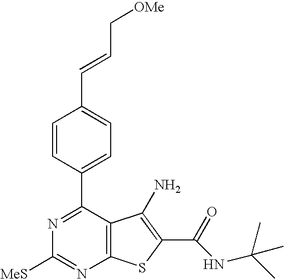

LogP: 4.44

CLogP: 5.3208

Compound 52/1 has the following structure:

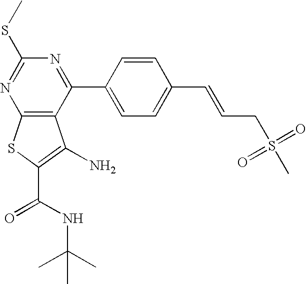

LogP: 3.08

CLogP: 3.8708

Compound 52/2 has the following structure:

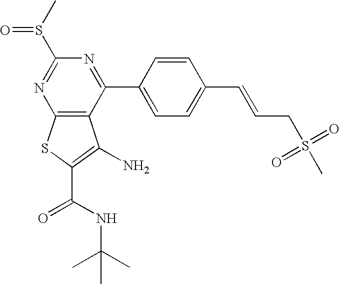

LogP: 1.3

CLogP: 1.71364

Compound 52/3 has the following structure:

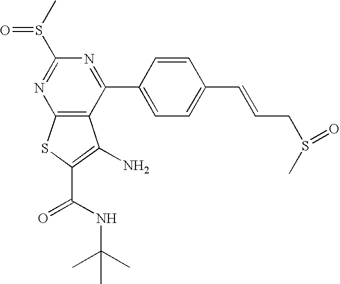

LogP: 1.26

CLogP: 1.83364

The synthesis of compound 52 was accomplished from a final step Suzuki coupling from the precursor brominated analogue (5-amino-4-(4-bromophenyl)-N-tert-butyl-2(methylthio)thieno[2,3-d]pyrimidine-6-carboxamide), which was synthesized according to methods reported in Moore et al., J Med Chem 49:3888-3896.

### Cell Culture and Transient Transfection

HEK-EM 293 cells were grown in Dulbecco's modified Eagle's Medium (DMEM) supplemented with 10% fetal bovine serum, 100 units/ml penicillin and 10 μg/ml streptomycin (Life Technologies Inc.) at 37° C. in a humidified 5% CO₂ incubator. Cells were transiently transfected with wild type TSHR and mutant receptors in 24-well plates (7.5×10⁴ cells per well) with 0.4 μg DNA/well using FuGENE™ 6 reagent (Roche) according to the manufacturer's protocol.

### Generation of Stable Cell-Lines Expressing TSHR, LHCGR or FSHR

The expression vectors for human TSHR and LHR are described in Jaschke et al., J Biol Chem 281:9841-9844. The FSHR cDNA in pcDNA3.1 was obtained from the Missouri S&T cDNA Resource Center (www.cDNA.org) and was subcloned into the pcDNA3.1(−)/hygromycin vector. HEK-EM 293 cells were transfected with the cDNA of TSHR, LHCGR or FSHR using FuGENE 6 Transfection reagent (Roche Diagnostics). Hygromycin (250 μg/ml) was used as selection marker.

### Site-Directed Mutagenesis of TSHR

The M9 mutant is described in Jaschke et al., J Biol Chem 281:9841-9844. The Y7.42A mutant was introduced into hTSHR-pcDNA3.1 via the QuickChange XL Site-Directed Mutagenesis kit (Stratagene). The construct was verified by sequencing (MWG Biotech).

### Determination of Intracellular Cyclic AMP Accumulation and Cell Surface Expression

Transiently transfected cells were cultured for 48 hours before the cAMP assay. HEK-EM 293 cells stably expressing TSHR, LHCGR or FSHR were seeded into 24-well plates with a density of 2.2×10⁵ cells/well 24 hours before the cAMP assay. After removal of growth medium, cells were incubated for 1 hour in HBSS (Cellgro) with 10 mM HEPES (Cellgro) containing 1 mM 3-isobutyl-1-methylxanthine (IBMX) (Sigma) and the ligand of interest in a humidified 5% CO₂ incubator at 37° C. The intracellular cAMP content was determined with the cAMP Biotrak Enzymeimmunoassay (EIA) System (GE Healthcare). Data were analyzed using GraphPad Prism 4 for Windows. Receptor expression was measured as described in Jaschke et al., J Biol Chem 281:9841-9844.

### Culture of Primary Human Thyrocytes

Thyroid tissue samples were obtained through the NIH Clinical Center during surgery for unrelated reasons. Patients provided informed consent on an IRB approved protocol and materials were received anonymously via approval of research activity through the Office of Human Subjects Research. The specimens were maintained in HBSS on ice and isolation of cells was initiated within 4 hours after surgery. All preparations were performed under sterile conditions. Tissue samples were minced into small pieces by fine surgical forceps and scissors in a 10 cm dish with a small volume of HBSS. Tissue pieces were transferred to a 15 ml tube (Falcon) and washed at least 3 times with HBSS. Afterward, tissue pieces were incubated with HBSS containing 3 mg/ml Collagenase Type IV (Gibco). Enzymatic digestion proceeded for 30 minutes or longer with constant shaking in a water bath at 37° C. until a suspension of isolated cells was obtained. After centrifugation for 5 minutes at 1000 rpm, the supernatant was removed and cells were resuspended in 10 ml DMEM with 10% FBS. Cells were plated in 10 cm tissue culture dishes and incubated at 37° C. in a humidified 5% CO₂ incubator. After 24 hours, the supernatant containing non-adherent cells was removed. The primary cultures of thyroid cells formed a confluent monolayer within 5-7 days. For determination of TPO mRNA expression, thyrocytes were seeded into 24-well plates at a density of 6×10⁴ cells/well 24 hours before the experiment.

### Compound 52 is a Selective Antagonist for TSHR

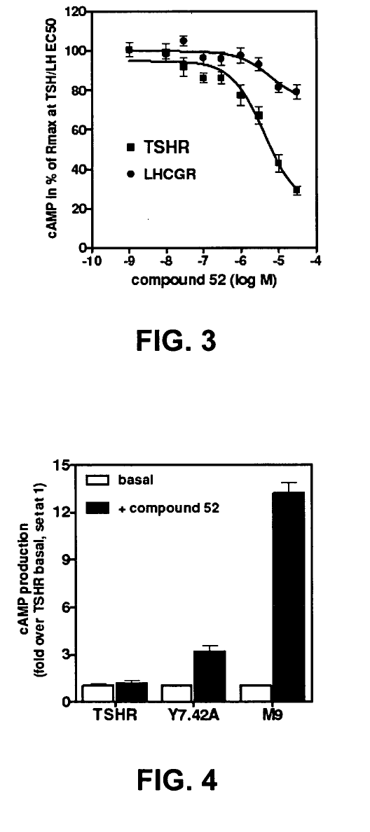

Compound 52 was found to be an antagonist for TSHR (FIG. 3) with no agonist activity (FIG. 4). The TSH-mediated cAMP response of TSHR was inhibited by a maximum of 70.8±5.5% at 30 μM compound 52. The IC₅₀ of compound 52 for TSHR inhibition is 4.2 μM (95% confidence interval: 2.3 μM-7.5 μM). In comparison, Org41841 is a partial agonist and inhibits TSH stimulation of the TSH receptor signaling but only by 35% and its IC₅₀ (with EC₅₀ dose of TSH) is 11 μM. Noteworthy, compound 52 is selective toward TSHR when compared to the closely related LHCGR and FSHR (FIG. 3). In contrast to TSHR, compound 52 is a partial agonist at LHCGR (17.25±2.25% activity compared to full activation of LHCGR by LH, set at 100%) (data not shown). Compound 52 has no activity at FSHR.

To exclude the possibility that compound 52 might be acting independently of TSHR by building aggregates with TSH, thereby inhibiting the TSH-induced response, tests were conducted for a possible aggregation between these two ligands. Compound 52 and TSH were preincubated for up to 15 minutes before addition to HEK-EM 293 cells stably expressing TSHR. Intracellular cAMP accumulation was determined in response to 30 μM compound 52 in the presence of 1.8 nM TSH (EC₅₀). There was no difference in the antagonistic effect whether TSH and compound 52 were preincubated together or not (data not shown) thereby excluding the possibility that its effect was caused by aggregation with TSH.

Evidence from Receptor Mutants for Interaction of Compound 52 with TSHR

Although compound 52 does not activate TSHR, it shows partial agonism at two TSHR mutants, one in which a tyrosine at position 7.42 in TMH7 was substituted by alanine (Y7.42A) and another, M9, in which nine residues in or near the Org41841 binding pocket were substituted by the corresponding residues of the LHCGR. These results are consistent with a hypothesis that compound 52 interacts with TSHR in the transmembrane domain. This hypothesis is supported by data that show that compound 52 does not compete with ¹²⁵I-labeled TSH for binding to TSHR.

The TSHR expresses high basal activity and, therefore, basal activity of mutants or ligand-stimulated activity of TSHR can be expressed as fold stimulation of this basal (constitutive) activity. Compound 52 stimulated cAMP production in HEK-EM 293 cells expressing these mutant receptors by 3.2±0.9-fold and 13.2±1.7-fold over TSHR basal activity of Y7.42A and M9, respectively (FIG. 4). Org41841, which is a partial agonist with 23% of TSH activity at TSHR, acts as a full agonist for M9, which can be explained by changes in hydrophobicity and gain of space at the three TMH/ECL junctions. Due to enlargement of the binding pocket of the chimeric M9 mutant compared to TSHR, compound 52 is located similarly to Org41841 in M9 and acts as an agonist. It is noteworthy that Y7.42A is constitutively active (1.73±0.38-fold over TSHR basal) even though it exhibits a cell surface expression of 74.90±8.04% compared to TSHR (data not shown). Because Y7.42A is sterically more relaxed than TSHR and the alanine is less bulky than tyrosine, compound 52 can move downward to the intracellular part of TMH6 and TMH7, as does Org41841. In this case the t-butyl group of compound 52 may sterically press apart the kinked TMH6 below P6.50 leading to a distinct TMH6 movement and activation of Y7.42A rather than antagonism observed in TSHR in which compound 52 sits higher in the binding pocket.

Inhibition of TSH Stimulation of Thyroperoxidase (TPO) mRNA Expression in Human Thyrocytes by Compound 52

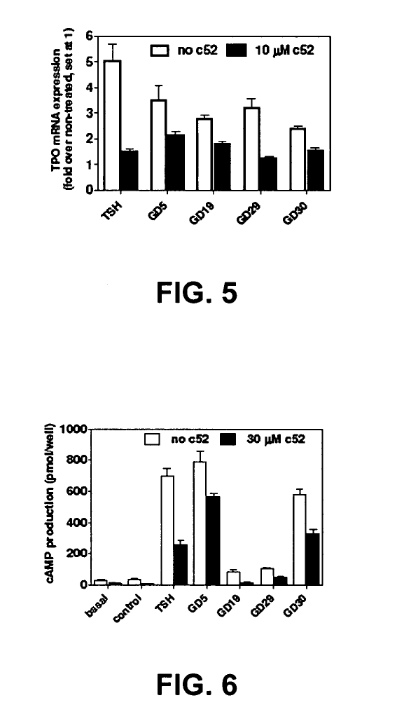

Normal thyroid tissue was received from two donors who underwent total thyroidectomy. Cells were either incubated with TSH or pretreated for 1 hour with compound 52 and then incubated with compound 52 in the presence of TSH for 24 hours. In thyrocytes from both donors, TSH alone increased TPO mRNA expression and this increase was inhibited by compound 52, both at 10 PM and 30 M compound (FIG. 5). In summary, compound 52 is an effective antagonist of TSH stimulation of endogenous TSHR activity in primary cultures of thyrocytes.

Compound 52 Inhibits TSHR Activation by Thyroid-Stimulating Antibodies (TsAbs) from Patients with Graves' Disease

To assess the therapeutic potential of compound 52 in patients with Graves' disease, its ability to inhibit TSHR activation by TsAbs was tested. First, four patient sera (GD 5, 19, 29, 30) were used at a dilution of 1:50 to test the effect of compound 52 on TsAb-stimulated cAMP accumulation in HEK-EM 293 cells expressing TSHR. All four sera increased cAMP accumulation, but to different extents (FIG. 6). To assess inhibition, cAMP accumulation was measured in response to TsAbs in the presence of 30 μM compound 52. Indeed, compound 52 reduced TsAb-mediated responses of the different sera (set at 100% for each patient's serum) by 28% to 79% (FIG. 6).

The inhibitory effect was confirmed of compound 52 on TsAb stimulation in primary cultures of human thyrocytes. TsAbs of all four patents' sera increased expression of TPO mRNA and addition of compound 52 inhibited TsAb-stimulated TPO mRNA expression for all sera tested (FIG. 5). This is an indication of the therapeutic potential of LMW antagonists.

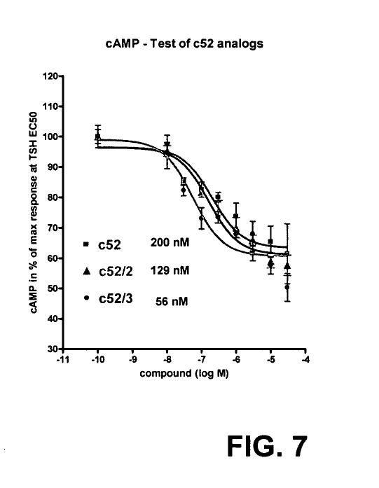

Compound 52/1 exhibited no antagonistic activity in a cAMP assay. Compounds 52/2 and 52/3 exhibit antagonist activity similar to compound 52 (see FIG. 7). Compounds 52/2 and 52/3 have improved solubility compared to compound 52.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound according to the formula 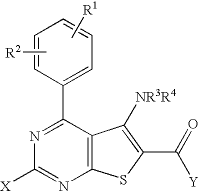
wherein X is —S(O)ₙR⁵;
n is 0, 1 or 2;
Y is —OR⁶ or —NR⁷R⁸
R¹ and R² independently are selected from optionally substituted lower aliphatic, alkoxy, aralkyl, halogen, H and —OR⁵, wherein R⁵ is selected from lower alkyl, H, aralkyl, acyl, alkoxycarbonyl and aminocarbonyl;
R³ and R⁴ independently are selected from acyl, alkoxycarbonyl, aminocarbonyl, aralkyl, H, lower alkyl and cycloalkyl;
R⁵ is selected from lower alkyl, aralkyl, cycloalkyl and haloalkyl;
R⁶ is selected from H, lower alkyl and aralkyl;
R⁷ and R⁸ independently are selected from H, lower alkyl, aralkyl and cycloalkyl; with the proviso that when R¹ is methoxy, R² is not H.

2. The compound of claim 1, according to the formula

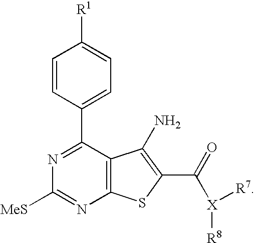

3. The compound of claim 1, according to the formula

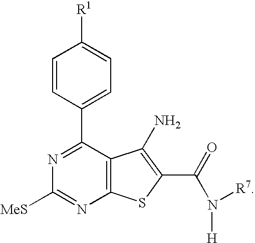

4. The compound of claim 3, wherein R⁷ is a sterically bulky alkyl group.

5. The compound of claim 1, according to the formula 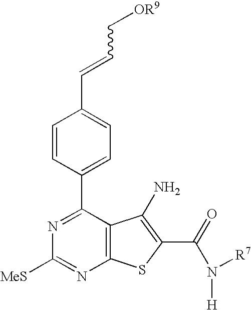
wherein R⁹ is selected from acyl, alkoxycarbonyl, aminocarbonyl, aralkyl, H, lower alkyl and cycloalkyl.

6. The compound of claim 5, wherein R⁹ is lower alkyl.

7. The compound of claim 5, according to the formula

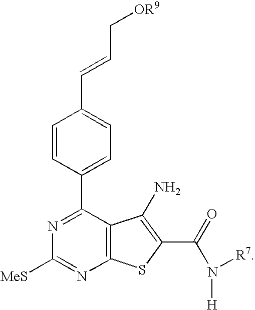

8. The compound of claim 5, according to the formula

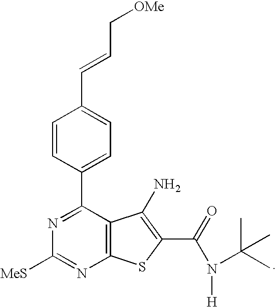

9. A pharmaceutical composition, comprising:
a pharmaceutically acceptable, carrier, adjuvant or vehicle; and a compound other than Org 41841 having the formula 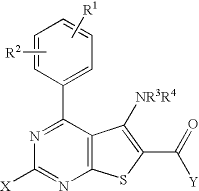
or any pharmaceutically acceptable salt thereof;
wherein X is —S(O)ₙR⁵;
n is 0, 1 or 2;
Y is —OR⁶ or —NR⁷R⁸
R¹ and R² independently are selected from optionally substituted lower aliphatic, alkoxy, aralkyl, halogen, H and —OR⁵, wherein R⁵ is selected from lower alkyl, H, aralkyl, acyl, alkoxycarbonyl and aminocarbonyl;
R³ and R⁴ independently are selected from acyl, alkoxycarbonyl, aminocarbonyl, aralkyl, H, lower alkyl and cycloalkyl;
R⁵ is selected from lower alkyl, aralkyl, cycloalkyl and haloalkyl;
R⁶ is selected from H, lower alkyl and aralkyl; and
R⁷ and R⁸ independently are selected from H, lower alkyl, aralkyl and cycloalkyl.

10. The pharmaceutical composition of claim 9, wherein the compound is a selective antagonist of the thyroid hormone receptor.

11. A method for treating a thyroid disorder, comprising providing a subject having a thyroid disorder and administering to the subject an effective amount of a compound of claim 1.

12. The method of claim 11, wherein the thyroid disorder is a hyperthyroid disorder.

13. The method of claim 12, wherein the hyperthyroid disorder is Graves' disease.

14. The method of claim 12, wherein the compound is a thyroid-stimulating hormone receptor antagonist.

15. The method of claim 14, wherein the compound has the formula

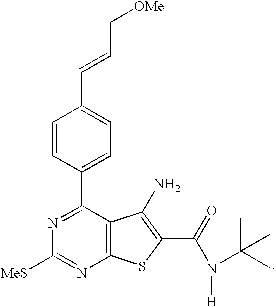

16. The method of claim 11, wherein the compound preferentially binds the thyroid-stimulating hormone receptor over the follicle-stimulating hormone receptor.

17. The compound of claim 3, wherein R¹ is a substituted lower aliphatic.

18. A compound, or a pharmaceutically acceptable salt thereof, according to the formula 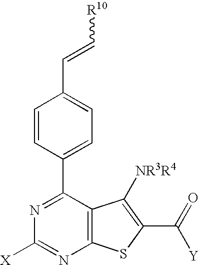
wherein R¹⁰ is —S(O)ₙR⁵ or —OR⁹, wherein R⁵ is selected from lower alkyl, H, aralkyl, acyl, alkoxycarbonyl and aminocarbonyl, n is 0, 1 or 2, and R⁹ is selected from acyl, alkoxycarbonyl, aminocarbonyl, aralkyl, H, lower alkyl and cycloalkyl;
X is —S(O)ₙR⁵; wherein R⁵ is selected from lower alkyl, H, aralkyl, acyl, alkoxycarbonyl and aminocarbonyl, and n is 0, 1 or 2;
Y is —OR⁶ or —NR⁷R⁸, wherein R⁶ is selected from H, lower alkyl and aralkyl, and R⁷ and R⁸ independently are selected from H, lower alkyl, aralkyl and cycloalkyl; and
R³ and R⁴ independently are selected from acyl, alkoxycarbonyl, aminocarbonyl, aralkyl, H, lower alkyl and cycloalkyl.

19. The compound of claim 18, wherein the compound has the formula

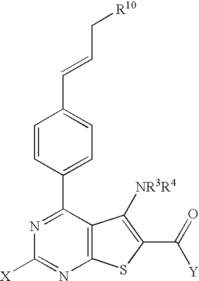

20. The compound of claim 19, wherein R¹⁰ is —S(O)ₙR⁵ and R⁵ is lower alkyl and n is 1 or 2; X is —S(O)ₙR⁵ and R⁵ is lower alkyl and n is 1 or 2; and Y is —NR⁷R⁸.

21. The compound of claim 20, wherein R⁷ and R⁸ are each independently H or lower alkyl, and R³ and R⁴ are each independently H or lower alkyl.

22. The compound of claim 19, wherein the compound has the formula

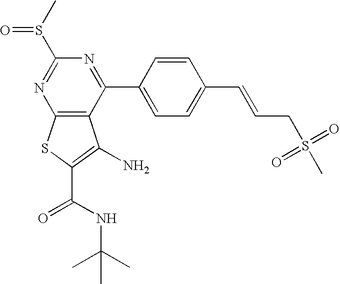

23. The compound of claim 19, wherein the compound has the formula

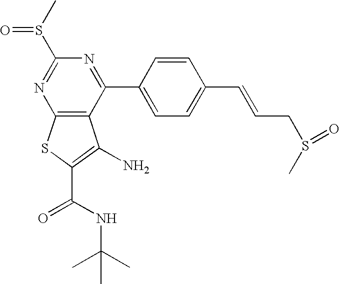

24. The compound of claim 20, wherein the compound is a thyroid stimulating hormone receptor antagonist.

25. A pharmaceutical composition comprising at least one compound of claim 18, and at least one pharmaceutically acceptable carrier.

26. A method for treating a thyroid disorder in a subject, comprising administering to the subject a therapeutically effective amount of at least one compound of claim 18.

27. A method for treating a thyroid disorder in a subject, comprising administering to the subject a therapeutically effective amount of at least one compound of claim 19.

28. The method of claim 27, wherein the thyroid disorder is a hyperthyroid disorder.

29. The method of claim 28, wherein the hyperthyroid disorder is Graves' disease.

30. The method of claim 26, wherein the compound is a thyroid-stimulating hormone receptor antagonist.

31. The compound of claim 8, wherein the compound is a selective thyroid-stimulating hormone receptor antagonist.

32. The compound of claim 8, wherein the compound exhibits no native thyroid-stimulating hormone receptor agonistic activity.
